(19) United States
(12) Patent Application Publication
(10) Pub. No.: US 2004/0122110 A1
(43) Pub. Date: Jun. 24, 2004

(54) Effect of vitamin A gel on paranasal sinus mucosal regeneration

(76) Inventors: Mendy S. MacCabee, Portland, OR (US); Peter H. Hwang, (US); Dennis R. Trune, Portland, OR (US)

Correspondence Address: DAVIS WRIGHT TREMAINE, LLP, 2600 CENTURY SQUARE, 1501 FOURTH AVENUE, SEATTLE, WA, 98101-1688, US

(21) Appl. No.: 10/658,962

(22) Filed: Sep. 8, 2003

Related U.S. Application Data
(60) Provisional application No. 60/408,792, filed on Sep. 6, 2002.

(51) Int. Cl. A61K 31/203; A61K 31/07

(52) U.S. Cl. 514/725; 514/559

(57) ABSTRACT

The present invention is directed, inter alia, to compositions and methods for treating, healing, or regeneration of ciliated epithelial structures, particularly those of mucosal membranes. In particular, the invention is directed to a method comprising topical administration of vitamin A (including retinoic acid) to ciliated epithelia structures, including, for example, the paranasal sinus mucosa, middle ear epithelium, and tracheal epithelium. In preferred embodiments the inventive methods are used in the context of surgery, and post-surgical healing and regeneration of epithelial and mucosal epithelial structures and cells.

### FIELD OF THE INVENTION

The present invention is directed, inter alia, to compositions and methods for treating, healing, or regeneration of ciliated epithelial structures, particularly those of mucosal membranes. In particular, the invention is directed to a method comprising topical administration of vitamin A (including retinoic acid) to ciliated epithelial structures, including, for example, the paranasal sinus mucosa, middle ear epithelium, and tracheal epithelium. In preferred embodiments the inventive methods are used in the context of surgery, and post-surgical healing and regeneration of epithelial and mucosal epithelial structures and cells.

### BACKGROUND OF THE INVENTION

The paranasal sinuses are lined by pseudostratified columnar ciliated epithelium that is responsible for clearing normal and infected sinus secretions. Ciliary transport is dependent on a number of complex interactions between the cilia and the sinonasal environment including nasal airflow, ostial patency, intranasal partial pressure of oxygen, humidity, temperature, mucous viscosity, and mucociliary structure and clearance rate. The speed of mucociliary transport averages 6 mm/min in humans and 10-15 mm/minute in rabbits.

Mucociliary clearance can be impaired by both infection and surgical trauma. For example, acute and chronic sinus disease may diminish ciliary function and regeneration because of increased fibrosis, decreased numbers of submucosal glands, and marked inflammatory changes. Additionally, paranasal sinus mucosa may suffer morphologic and functional alterations as a result of surgical trauma.

Despite recent refinements in mucosal-preserving surgical technique, inadvertent stripping of sinus mucosa is often unavoidable. Regenerated mucosa from surgically stripped sinuses has shown ultrastructural changes such as fibrosis, compound cilia, stripped sinuses has shown ultrastructural changes such as fibrosis, compound cilia, inflammatory infiltrate, dysmorphic or absent cilia, bleb formation, abnormal microtubule formation, and compromised mucociliary function.

There is a strong need in the art for a novel method to promote healing or regeneration of stripped ciliated epithelium, including tracheal, paranasal and middle-ear epithelia, with better function and reduced morbidity.

There is a pronounced need in the art for a novel method to preserve mucosal integrity and function in patients who have undergone surgery for chronic infection or require medical management for chronic sinus disease.

There is a pronounced need in the art for a novel method to reduce fibrosis in the context of stripped ciliated epithelium, or in patients who have undergone surgery for chronic infection or require medical management for chronic sinus disease.

There is a pronounced need in the art for a novel method to promote sinus wound healing.

These and other limitations and problems of the past are solved by the present invention.

### SUMMARY OF THE INVENTION

According to embodiments of the present invention, topical vitamin A administration enhances regeneration of damaged ciliated epithelium including that of paranasal sinus mucosa. Topical vitamin A administration has general utility for promoting sinus wound healing including sinus mucosa healing and function after chronic sinus disease or surgery.

The invention will best be understood by reference to the following detailed description of the preferred embodiment, taken in conjunction with the accompanying drawings. The discussion below is descriptive, illustrative and exemplary and is not to be taken as limiting the scope defined by any appended claims.

### BRIEF DESCRIPTION OF THE DRAWINGS

FIG. 1 shows a tissue specimen from normal, non-stripped control paranasal sinus mucosa.

FIG. 2 shows a tissue specimen from stripped, untreated paranasal sinus mucosa.

FIG. 3 shows a tissue specimen of healed paranasal sinus mucosa from a low-concentration vitamin A (about 0.01% retinoic acid) group.

FIG. 4 shows a tissue specimen of healed paranasal sinus mucosa from a high-concentration vitamin A (about 0.025% retinoic acid) group.

FIG. 5 shows a scanning electron micrograph (EM) of normal rabbit sinus epithelium, 2000×.

FIG. 6 shows a scanning EM of rabbit sinus epithelium at 14 days after surgical stripping with no additional treatment, 2000×.

FIG. 7 shows a scanning EM of rabbit sinus epithelium at 14 days after surgical stripping and treatment with gel control (minus vitamin A), 2000×.

FIG. 8 shows a scanning EM of rabbit sinus epithelium at 14 days after surgical stripping and treatment with Vitamin A gel, 2000×.

### DETAILED DESCRIPTION OF THE INVENTION DEFINITIONS

The term “ciliated epithelium” refers generally to all ciliated epithelial structures, and in preferred embodiments refers to that of sinus and paranasal sinus, tracheal, and middle-ear ciliated epithelium.

The term Middle-ear epithelium refers to that as described, for example, by Wenig, B. M., Atlas of Head and Neck Pathology, Saunders 1993, page 341: Middle ear: Lining is composed of respiratory epithelium, varying from ciliated epithelium (eustachian tube) to a flat, single, cuboidal epithelium.

The term “Vitamin A” refers generally to C₂₀-β-ionone derivatives that exhibit qualitatively the biological activity of all-trans retinol, and also encompasses retinoids and retinoic acid, and compounds that control epithelial differentiation and prevent metaplasia, without possessing the full range of activities of vitamin A. In preferred embodiments herein, retinoic acid is used (see Weber, F. & Cornish-Bowden, A., Vitamin A and retinoids, Br. J Nutr. 74, 869-870,1995).

Topical Vitamin A (Including Retinoic Acid) Was Demonstrated to Have Therapeutic Utility for Promoting Regeneration and Healing of Damaged Ciliated Epithelial Structures Including, for Example That of Paranasal Sinus Mucosa

Paranasal sinus wound healing occurs in a few well-defined phases. Mucous membrane healing occurs by the migration of cells from normal adjacent epithelium, followed by multiplication and differentiation of progenitor cells. Epithelial regeneration begins within a few hours of the insult at an estimated velocity of 4-20 micrometers per hour. Sinus mucosa basal cells are multipotent with the ability to differentiate into squamous, ciliated, and goblet cells. Undifferentiated basal cells appear to be the main source of new progenitor cells in paranasal sinus mucosa. Co-factors influencing the differentiation of ciliated cells have not been well defined.

Vitamin A is believed to regulate cellular proliferation and differentiation of epithelial tissues. The systemic deficiency of vitamin A in experimental animals leads to the development of squamous metaplasia. Metaplasia in respiratory tract epithelium results from proliferation of basal cells and their subsequent transformation into squamous keratinizing cells instead of goblet and ciliated cells. The systemic administration of vitamin A (including retinoic acid) has been shown to aid in regeneration of normal ciliated tracheal epithelium in systemically deprived hamsters.

Vitamin A is thought to regulate replication of basal cells and therefore ciliated progenitor cells. It also modulates the replication of mucous cells, which are essential for generation of the mucus layer necessary for proper mucociliary transport function. In McDowell's study with hamster tracheal upper respiratory epithelium, preciliated cells were virtually absent in the systemically deprived vitamin A group (McDowell et al., Virchows Arch [Cell Pathol] 45: 221-240, 1884; see also Chopra et al., Cell Tissue Kinet 23:575-586, 1990). With restoration of systemic vitamin A levels, ciliated progenitor cells rapidly developed cilia and further matured into functional ciliated epithelium. Additionally, in a study by Edmondson et al, systemic vitamin A deprivation in hamsters resulted in squamous metaplasia of pseudostratified ciliated tracheal epithelium with loss of goblet cells, resulting in loss of mucus secreting capability and an overall disruption of the mucociliary microenvironment (Edmondson et al., J Cell Physiol 142:21-30, 1985).

Patients with chronic sinus disease often have inflamed, polypoid mucosa that can be easily stripped away during functional endoscopic sinus, even when great care is taken to preserve the mucosal lining.

Examples of relevant sinus surgeries are, but are not limited to, Adenoidectomy; Endoscopic Sinus Surgery; Ethmoidectomy; Extended Endscopic Frontal Sinus Surgery; Frontal Sinusotomy; FESS (Functional Endoscopic Sinus Surgery); Maxillary Sinusotomy; Open Frontal Sinus Surgery; Polypectomy; Reduction Removal of Inferior Turbinate; Reduction Removal of Middle Turbinate; Septoplasty; Sphenoidotomy; Tumor Removal.

According to the present invention, non-systemic vitamin A administration restores, inter alia, ciliated paranasal sinus epithelium and mucosal structure.

Pharmaceutical Formulations. Compositions of vitamin A (including retinoic acid), as taught and disclosed herein can be used to treat, heal, or otherwise regenerate ciliated epithelial structures, particularly those of mucosal membranes, tracheal epithelium and middle-ear epithelium. The particular method, formulation and mode of administration will depend upon the therapeutic indication, and the physico-chemical properties of the formulation, and the target organ or tissue. Different approaches are discussed.

Non-systemic (e.g., topical) administration may be accomplished by the administration of aqueous gels, solutions, ointments, salves, gels, lotions, unguents, sprays and aerosolized or nebulized particles, and/or coatings and/or impregnation of packing material such as but not limited to sponge material and strip gauze, or the like containing vitamin A to the epithelium in question, for example, the paranasal sinus mucosa. Techniques for the formulation and administration of the compounds of the instant application are found, for example, in “Remington's Pharmaceutical Sciences” Mack Publishing Co., Easton, Pa., latest edition. Pharmaceutical compositions for use in accordance with the present invention are formulated in a conventional manner using one or more physiologically acceptable carriers or excipients.

Additionally, the compounds may also be formulated as a depot preparation. Such long-acting formulations may be administered by implantation or packing. Thus, for example, the compounds may be formulated with suitable polymeric or hydrophobic materials (e.g., as an emulsion in an acceptable oil) or ion-exchange resins, or as sparingly soluble derivatives, for example, as a sparingly soluble salt. Examples of pharmaceutically acceptable salts, carriers or excipients are well known to those skilled in the art and can be found, for example, in Remington's Pharmaceutical Sciences, 18th Edition, A. R. Gennaro, Ed., Mack Publishing Co., Easton, Pa., 1990. Such salts include, but are not limited to, sodium, potassium, lithium, calcium, magnesium, iron, zinc, hydrochloride, hydrobromide, hydroiodide, acetate, citrate, tartrate, malate salts, and the like.

The inventive compositions and formulations can be administered in conjunction with another therapy or drug.

Dose Determinations. Toxicity and therapeutic efficacy of the inventive compositions and formulations are determined by standard pharmaceutical and toxicologic procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred. While compounds that exhibit toxic side effects may be used, care should be taken to design a delivery system that targets such compounds to the desired site or affected tissue in order to minimize potential damage to other cells and, thereby, reduce side effects.

Data obtained from cell culture assays and animal studies is used in formulating a range of dosage for use in humans. The dosage of such compounds lies preferably within a range of concentrations that include the ED₅₀ with little or no toxicity. The dosage may vary within this range depending upon the dosage form employed and the route of administration utilized. For any compound used in the methods of the invention, the therapeutically effective dose is typically estimated initially from cell culture or animal model assays. A dose may be formulated in animal models to achieve a concentration range that includes the IC₅₀ (i.e., the concentration of the test compound that achieves a half-maximal inhibition of symptoms). Such information is used to more accurately determine useful doses in humans.

A therapeutically effective dose further refers to that amount of the compound sufficient to promote ciliated epithelial healing or regeneration, such as in paranasal sinus mucosa.

Alternately, a therapeutically effective dose refers to that amount of the compound sufficient to reduce serous gland loss.

Alternatively, a therapeutically effective dose refers to that amount of the compound sufficient to reduce laminar fibrosis, such as within the lamina propria.

Alternatively, a therapeutically effective dose refers to that amount of the compound sufficient to affect a mucociliary density change, or a greater density of regenerated cilia.

Alternatively, a therapeutically effective dose refers to that amount of the compound sufficient to affect bone morphometry, such and sinus bone morphometry.

Preferably, vitamin A (including retinoic acid) is administered at about 0.001% to about 0.25% (w/w). Preferably, vitamin A (including retinoic acid) is administered at about 0.005% to about 0.025% (w/w). Preferably, vitamin A (including retinoic acid) is administered at about 0.01% to about 0.025% (w/w). Preferably, vitamin A (including retinoic acid) is administered at about 0.001% to about 0.05% (w/w).

Therapeutically effective doses are administered alone or as adjunctive therapy in combination with other treatments.

The amount of therapeutically active compounds that are administered and the dosage regimen for treating with the compounds and/or compositions of this invention depends on a variety of factors, including the age, weight, sex and medical condition of the subject, the severity of the disease, the route and frequency of administration, and the particular compound employed, and thus may vary widely.

The daily dose can be administered in one to multiple doses per day, or may be in depot formulation.

Topical ointments, creams, or suppositories or packings containing the active ingredients. When formulated in an ointment, the active ingredients may be employed, e.g., with either paraffinic or a water-miscible ointment base. Alternatively, the active ingredients may be formulated in a cream with an oil-in-water cream base. If desired, the aqueous phase of the cream base may include, for example at least 30% w/w of a polyhydric alcohol such as propylene glycol, butane-1,3-diol, mannitol, sorbitol, glycerol, polyethylene glycol and mixtures thereof. The topical formulation may desirably include a compound which enhances absorption or penetration of the active ingredient through the skin or other affected areas. Examples of such dermal penetration enhancers include dimethylsulfoxide and related analogs. The compounds of this invention can also be administered by a nebulizing or aerosolizing device.

Preferably, topical administration can be accomplished using a patch or packing either of the reservoir and porous membrane type or of a solid matrix variety. In either case, the active agent is delivered continuously from the reservoir or microcapsules through a membrane into the active agent permeable adhesive, which is in contact with the skin or mucosa of the recipient.

The oily phase of the emulsions of this invention may be constituted from known ingredients in a known manner. While the phase may comprise merely an emulsifier, it may comprise a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make-up the so-called emulsifying wax, and the wax together with the oil and fat make up the so-called emulsifying ointment base which forms the oily dispersed phase of the cream formulations. Emulsifiers and emulsion stabilizers suitable for use in the formulation of the present invention include Tween 60, Span 80, cetostearyl alcohol, myristyl alcohol, glyceryl monostearate, and sodium lauryl sulfate, among others.

The choice of suitable oils or fats for the formulation is based on achieving the desired properties, since the solubility of the active compound in most oils likely to be used in pharmaceutical emulsion formulations is very low. Thus, the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters may be used. These may be used alone or in combination depending on the properties required. Alternatively, high melting point lipids such as white soft paraffin and/or liquid paraffin or other mineral oils can be used.

For therapeutic purposes, the active compounds of this combination invention may be combined with one or more adjuvants appropriate to the indicated route of administration. The compounds may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets may contain a controlled-release formulation as may be provided in a dispersion of active compound in hydroxypropylmethyl cellulose.

Other formulations may be in the form of aqueous or non-aqueous isotonic sterile injection solutions or suspensions. These solutions and suspensions may be prepared from sterile powders or granules having one or more of the carriers or diluents mentioned for use in the formulations for oral administration. The compounds may be dissolved in water, polyethylene glycol, propylene glycol, ethanol, corn oil, cottonseed oil, peanut oil, sesame oil, benzyl alcohol, sodium chloride, and/or various buffers. Other adjuvants and modes of administration are well and widely known in the pharmaceutical art.

When administered, the pharmaceutical preparations of the invention are applied in pharmaceutically-acceptable amounts and in pharmaceutically-acceptably compositions. Such preparations may routinely contain salt, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrocholoric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The compositions may be combined, optionally, with a pharmaceutically-acceptable carrier. The term “pharmaceutically-acceptable carrier” as used herein means one or more compatible solid or liquid filler, diluents or encapsulating substances which are suitable for administration into a human or other animal. The term “carrier” denotes an organic or inorganic ingredient, natural or synthetic, with which the active agent is combined to facilitate the application. The components of the osmotic agents and the active agents also are capable of being co-mingled with such carriers, other additives, and with each other, in a manner such that there is no interaction which would substantially impair the desired pharmaceutical efficacy. The term “vehicles” encompasses impregnated packings and the like.

The pharmaceutical compositions may contain suitable buffering agents, including: acetic acid in a salt; citric acid in a salt; boric acid in a salt; and phosphoric acid in a salt.

The pharmaceutical compositions may conveniently be presented in unit dosage form and may be prepared by any of the methods well known in the art of pharmacy. All methods may include the step of bringing the active agents and osmotic agents into association with a carrier which constitutes one or more accessory ingredients. In general, the compositions may be prepared by uniformly and intimately bringing the active agents into association with a liquid carrier, a finely divided solid carrier, or both, and then, if necessary, shaping the product.

### EXAMPLE 1

### (In vivo application of Vitamin A to paranasal sinus of rabbits resulted in epithelial healing of the sinus mucosa)

The effects of topical vitamin A gel on regeneration of paranasal sinus mucosa in mechanically denuded rabbit sinuses were studied and observed herein. New Zealand White Rabbits were used in these experiments. The New Zealand White Rabbit model system is an art-recognized sinus model for humans, because rabbit maxillary sinuses are large and easily accessible, and the immune and healing response closely mimics that observed in humans.

Untreated, regenerated mucosa showed expected changes of submucosal gland loss, basal lamina and lamina propria fibrosis, cellular atypia, and loss of cilia. Application of vitamin A gel topically resulted in substantially more normal mucosal regeneration marked by less cellular atypia and fibrosis. Although the regenerated mucosa had some cellular abnormalities, the mucocilliary blanket was near normal in appearance. The group receiving the lower concentration of vitamin A (about 0.01% w/v) had more favorable morphology than the higher concentration (0.025% w/v) vitamin A group, and both were improved when compared to no treatment.

Methods

Surgery. The animals were anesthetized with an intramuscular injection of rabbit cocktail 1 ml/kg (1 ml acepromazine maleate 10 mg/ml+2.5 ml Xylazine 20 mg/ml+5 ml ketamine 100 mg/ml). They were then intubated and surgical anesthesia maintained with isofluorane inhalant anesthesia throughout the operative procedure. A midline nasal dorsum incision was made and skin flaps elevated laterally to expose the face of the maxillary sinus. The anterior wall of the left and right maxillary sinuses was removed with a drill and cutting burr. The opening was enlarged as needed with a Kerrison rongeur until the entire anterior bony wall was removed; all other bony walls were undisturbed. The entire mucosal lining of the maxillary sinus was removed en bloc with a Rosen elevator, and any remaining mucosal lining was removed with a curette. The mucosa overlying the natural ostium was intentionally preserved to ensure patency of the sinus outflow tract. Following the respective treatment described herein below the overlying periosteum was closed with a running 3-0 vicryl suture. The skin was closed with a running, subcutaneous 3-0 vicryl suture and the animals allowed to heal for fourteen days. All animals received three days of oral antibiotic therapy with Enrofloxacin 22.7 mg/ml given intramuscularly at 2.5 mg/kg/day. Postoperative pain was controlled with oral Buprenorphine (0.3 mg/ml) 0.3-0.6 mg/kg intramuscularly every six hours as needed for pain. There were no surgical complications and no postoperative wound dehiscences or infections.

Treatment. Both left and right maxillary sinuses were opened and stripped of mucosa in twelve (12) New Zealand white rabbits of either sex and body weight of 2.5-4.0 kg. Six of the left sinuses were treated with a solution of about 0.01% vitamin A in an aqueous gel (low concentration group), and the remaining six with about 0.025% topical vitamin A in an aqueous gel (high concentration group). The right maxillary sinuses of these twelve rabbits were stripped and no treatment was applied. Care was taken not to overfill the sinus cavities in order to prevent cross contamination between treated and untreated sinuses. The right maxillary sinuses of these twelve rabbits served as the untreated control group to reflect the normal healing process. Each rabbit served as its own control. Six additional animals had their right maxillary sinuses opened but otherwise unaltered to demonstrate normal sinus mucosa morphology. The sinus mucosa was examined by light microscopy after 14 days.

Tissue Preparation. On postoperative day fourteen, the animals were sacrificed with intracardiac pentobarbital (85 mg/kg) in Euthasol™, available from Delmarva Laboratories, Incorporated, Virginia; a commercial euthanasia solution of pentobarbital sodium and phenytoin sodium. The maxillary sinuses were reopened through the prior incisions and the medial wall of the sinus cavities harvested, taking care to preserve the overlying regenerated mucosa. The underlying bone and mucosa were fixed immediately in 3% glutaraldehyde and 1.5% paraformaldehyde in 0.1 M phosphate buffer for twenty-four hours. The tissue was decalcified in 10% EDTA-TRIS buffer for fourteen days, embedded in glycol methacrylate (GMA) plastic, sectioned at three microns, stained with methylene blue and basic fuchsin, and examined with the light microscope. Individual sections were evaluated for gross ciliary morphology, degree of ciliary loss, fibrosis of the basal lamina and lamina propria, overall goblet cell to ciliated cell ratio, serous gland presence, and neo-osteogenesis of the underlying bone. The initial tissue evaluation was performed and recorded by two separate, unbiased observers. Multiple sources were used for histologic and pathologic reference.

Electron micrographs of the respective tissues were prepared using standard methods, well known in the art. Briefly, specimens were affixed to a brass coupon and dipped in liquid nitrogen via a threaded rod. Specimens were then processed on a cryostage to sublimate any surface water at −80° C. for 3-5 minutes under high vacuum. Samples were then gold coated to a thickness of 10-20 nm at −140° C., then imaged by scanning electron microscopy.

Results

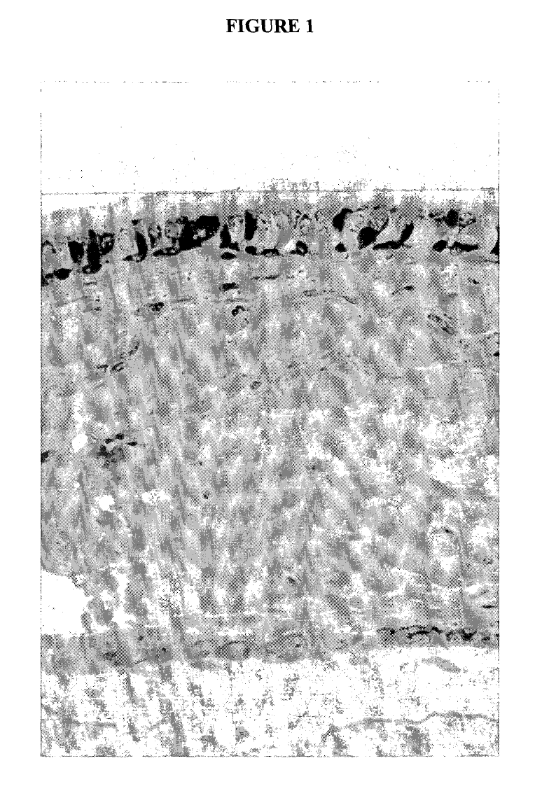

1. Normal, Non-stripped Controls. As shown in FIG. 1, normal control tissue specimens showed histology comparable to that of normal rabbit maxillary sinus. Two or three layers of pseudostratified ciliated epithelium were identified, with ciliated, goblet and basal cells on the basal lamina membrane. Ciliated cells outnumbered other cell types. The lamina propria, located below the basal membrane, contained numerous serous glands and vessels.

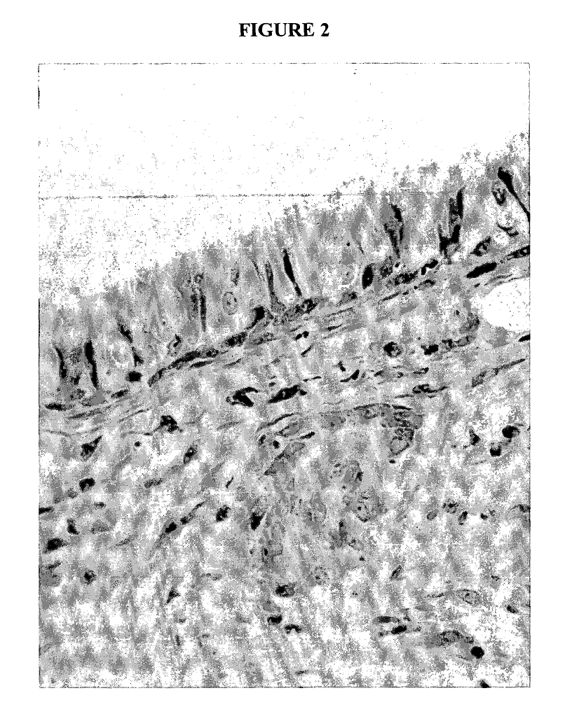

2. Stripped, Untreated Controls. Compared to normals, the stripped control group showed loss of the submucosal serous gland layer, but significant fibrosis of the basal lamina and lamina propria. As shown in FIG. 2, ciliary density was markedly diminished with few tufts of cilia surrounded predominantly by denuded mucosal segments.

3. Treated Groups. Overall, topical vitamin A treatment, both high and low concentrations, of stripped paranasal sinus mucosa substantially improved mucosal and ciliary regeneration.

Treated sinuses were marked by less cellular atypia and fibrosis compared to the stripped, untreated control group. The low concentration vitamin A group had basal lamina fibrosis and loss of submucosal serous glands, but a near normal mucociliary blanket qualitatively. The high concentration vitamin A group showed more histologic atypia and heterogeneity in the cellular layer when compared to the low concentration and normal groups. However, the high concentration vitamin A treatment had less reactive fibrosis in the basal lamina and lamina propria and substantially more normal mucociliary blanket coverage when compared to stripped, untreated controls.

In terms of ciliary morphology and density, the vitamin A groups showed cilia that were grossly normal in appearance but slightly diminished in number compared to the normal, non-stripped controls. There were no frankly denuded segments in the vitamin A treatment groups. When the high and low concentration groups were compared to each other, the low concentration group had a greater density of regenerated cilia.

In terms of other epithelial features, both vitamin A groups had an increased goblet cell to ciliated cell ratio compared to normals. This goblet cell hyperplasia was also noted in the stripped control group. Both vitamin A groups had increased fibrosis of the basal lamina and lamina propria when compared to normals. However, there was substantially less fibrosis in the vitamin A treatment groups than in the stripped, untreated controls.

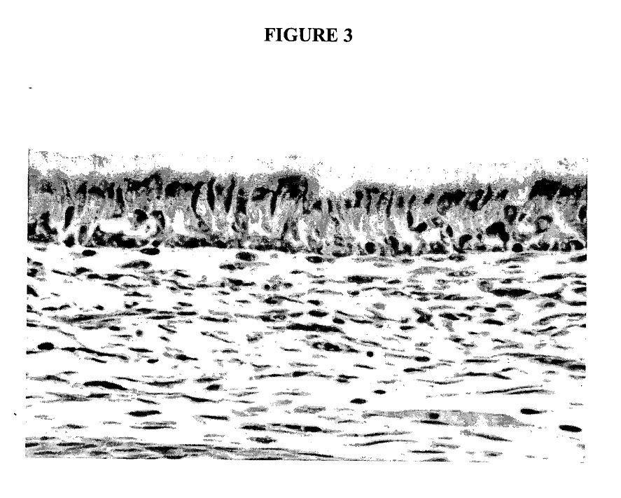

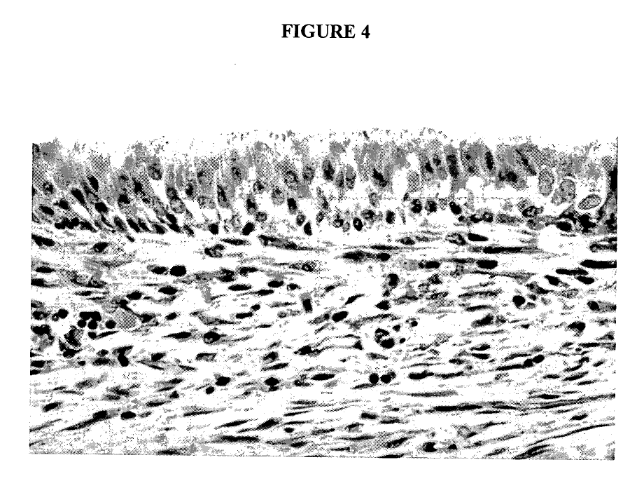

Both vitamin A groups and the stripped control group had a relative loss of the serous gland layer. Morphologically, the regenerated mucosa in the vitamin A treatment groups was still grossly abnormal, although the degree of cellular and ciliary abnormality was markedly diminished as shown in FIGS. 3 and 4 when compared to stripped controls as shown in FIG. 2.

Significantly, changes within each treatment group, as well as the control groups, were uniform across each group and were consistently seen in all samples within each treatment group. The lower concentration vitamin A group qualitatively had more favorable morphology than the higher concentration vitamin A group. However, the higher concentration group was still dramatically improved when compared to the stripped, untreated control group.

In the experiments corresponding to FIGS. 1-4, there was not a control group that received a non-medicated aqueous gel to determine whether any observed changes associated with vitamin A are potentially confounded by possible beneficial moisturizing effects of the gel vehicle. However, in additional and further experiments conducted with such a control group that did receive a non-medicated aqueous gel, the results presented herein were entirely confirmed (see electron micrographs below). Moreover, in the present experiments, the observed dose-response sensitivity of mucosa to varying concentrations of vitamin A confirms the positive effects of vitamin A.

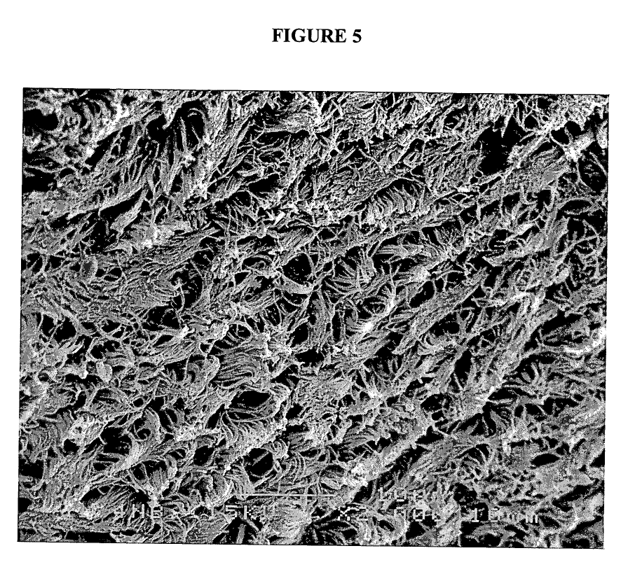

FIG. 5 shows a scanning electron micrograph (EM) of normal rabbit sinus epithelium (such as that of FIG. 1), 2000×.

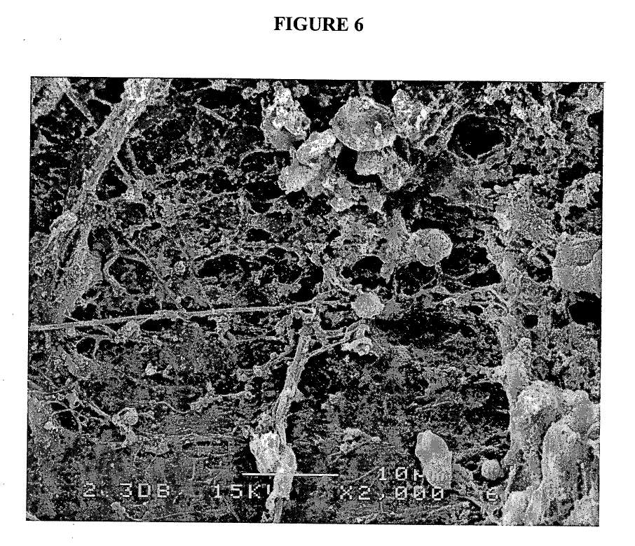

FIG. 6 shows a scanning EM of rabbit sinus epithelium at 14 days after surgical stripping with no additional treatment (such as that of FIG. 2), 2000×.

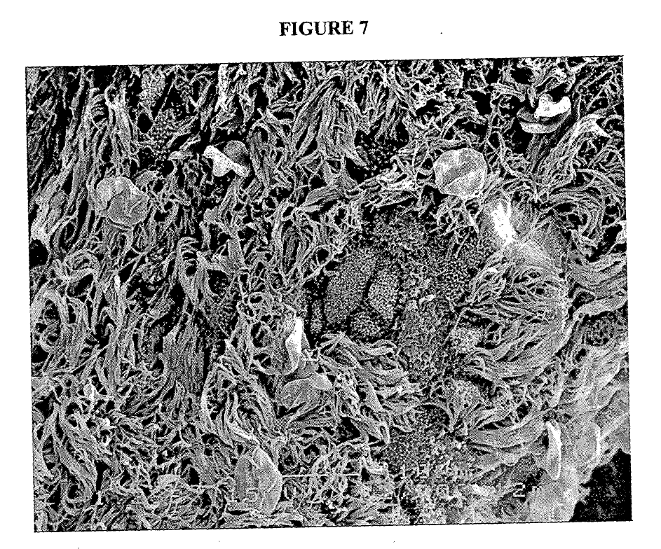

FIG. 7 shows a scanning EM of rabbit sinus epithelium at 14 days after surgical stripping and treatment with gel control (minus vitamin A) (such as that of FIG. 3), 2000×.

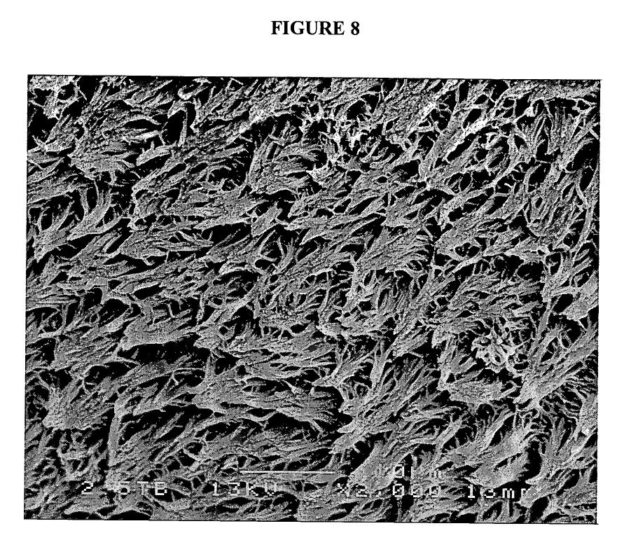

FIG. 8 shows a scanning EM of rabbit sinus epithelium at 14 days after surgical stripping and treatment with Vitamin A gel (such as that of FIG. 4), 2000×.

The discussion above is descriptive, illustrative and exemplary and is not to be taken as limiting the scope defined by any appended claims.

We claim:

1. A method for treating a damaged ciliated epithelial structure, comprising topical administration of a therapeutically effective amount of a composition comprising vitamin A, whereby treating of the damaged ciliated e epithelial structure is, at least in part, achieved.

2. The method of claim 1, wherein the ciliated epithelial structure is selected from the group consisting of: nasal or paranasal sinus mucosa; tracheal epithelium; middle-ear epithelium, including respiratory epithelium, ciliated epithelium and cuboidal epithelium; and combinations thereof.

3. The method of claim 1, wherein the ciliated epithelial structure comprises ciliated paranasal sinus mucosa.

4. The method of claim 1, wherein damage comprises damage selected from the group consisting of acute or chronic sinus disease, infection, mechanical, surgical intervention, and combinations thereof.

5. The method of claim 1, wherein damage is that caused by surgical intervention.

6. The method of claim 1, wherein topical administration comprises administration by a medium selected from the group consisting of aqueous or non-aqueous gels, solutions, ointments, salves, lotions, unguents, sprays, aerosolized or nebulized particles, coatings, impregnations of packing material or sponge material or strip gauze, and combinations thereof.

7. The method of claim 1, wherein treating comprises affecting an indicator selected from the group consisting of: increase, relative to untreated, in ciliated paranasal sinus mucosa; promotion, relative to untreated, of ciliated epithelial healing or regeneration; reduction, relative to untreated, of serous gland loss; reduction, relative to untreated, of laminar fibrosis, including of the lamina propria; effect, relative to untreated on mucociliary density change, including causing a greater density of regenerated cilia; effect, relative to normal, on bone morphometry, including sinus bone morphometry.

8. The method of claim 1, wherein vitamin A is administered at a concentration range selected from the group consisting of: about 0.001% to about 0.25% (w/w); about 0.005% to about 0.025% (w/w); about 0.01% to about 0.025% (w/w), and about 0.001% to about 0.05%.

9. A pharmaceutical composition for topically treating a damaged ciliated epithelial structure, comprising a therapeutically effective amount of vitamin A and at least one of a pharmaceutically acceptable diluent, excipient, or vehicle, to be administered topically to damaged ciliated epithelial structures.

10. The pharmaceutical composition of claim 9, wherein vitamin A is at a concentration range selected from the group consisting of: about 0.001% to about 0.25% (w/w); about 0.005% to about 0.025% (w/w); about 0.01% to about 0.025% (w/w), and about 0.001% to about 0.01% (w/w).

11. A method for treating a damaged ciliated epithelial structure, comprising topical administration of a therapeutically effective amount of a composition comprising vitamin A (including retinoic acid), wherein the ciliated epithelial structure is selected from the group consisting of nasal or paranasal sinus mucosa, tracheal epithelium, middle-ear epithelium, and combinations thereof, and whereby treating of the damaged ciliated epithelial structure is, at least in part, achieved.

12. The method of claim 11, wherein topical administration comprises administration by a medium selected from the group consisting of aqueous or non-aqueous gels, solutions, ointments, salves, lotions, unguents, sprays, aerosolized or nebulized particles, coatings, impregnations of packing material or sponge material or strip gauze, and combinations thereof.

13. Use, topically, of vitamin A in manufacture of a medicament for the treatment of damaged ciliated epithelial structures.

14. The use of claim 13, wherein the ciliated epithelial structure is selected from the group consisting of: nasal or paranasal sinus mucosa; tracheal epithelium; middle-ear epithelium, including respiratory epithelium, ciliated epithelium and cuboidal epithelium; and combinations thereof.

15. The use of claim 13, wherein the ciliated epithelial structure comprises ciliated paranasal sinus mucosa.

16. The use of claim 13, wherein damage comprises damage selected from the group consisting of acute or chronic sinus disease, infection, mechanical, surgical intervention, and combinations thereof.

17. The use of claim 13, wherein damage is that caused by surgical intervention.

18. The use of claim 13, wherein topical administration comprises administration by a medium selected from the group consisting of aqueous or non-aqueous gels, solutions, ointments, salves, lotions, unguents, sprays, aerosolized or nebulized particles, coatings, impregnations of packing material or sponge material or strip gauze, and combinations thereof.

19. The use of claim 13, wherein treating comprises affecting an indicator selected from the group consisting of: increase, relative to untreated, in ciliated paranasal sinus mucosa; promotion, relative to untreated, of ciliated epithelial healing or regeneration; reduction, relative to untreated, of serous gland loss; reduction, relative to untreated, of laminar fibrosis, including of the lamina propria; effect, relative to untreated on mucociliary density change, including causing a greater density of regenerated cilia; effect, relative to normal, on bone morphometry, including sinus bone morphometry.

20. The use of claim 13, wherein vitamin A is administered at a concentration range selected from the group consisting of: about 0.001% to about 0.25% (w/w); about 0.005% to about 0.025% (w/w); about 0.01% to about 0.025% (w/w), and about 0.001% to about 0.05%.

21. The method of any one of claims 1 or 11, wherein vitamin A is retinoic acid.

22. The pharmaceutical composition of claim 9, wherein vitamin A is retinoic acid.

23. The use of claim 13, wherein vitamin A is retinoic acid.
